(19) United States
(12) Patent Application Publication
(10) Pub. No.: US 2023/0242880 A1
(43) Pub. Date: Aug. 3, 2023

(54) METHOD OF INDUCING REGULATORY T CELLS

(71) Applicants: PUBLIC UNIVERSITY CORPORATION NAGOYA CITY UNIVERSITY, Nagoya-city, Aichi (JP); USHIO DENKI KABUSHIKI KAISHA, Tokyo (JP)

(72) Inventors: Akimichi MORITA, Aichi (JP); Hideyuki MASUDA, Tokyo (JP)

(73) Assignees: PUBLIC UNIVERSITY CORPORATION NAGOYA CITY UNIVERSITY, Nagoya-city, Aichi (JP); USHIO DENKI KABUSHIKI KAISHA, Tokyo (JP)

(21) Appl. No.: 18/002,651

(22) PCT Filed: May 11, 2021
(86) PCT No.: PCT/JP2021/017851

§ 371 Date: Dec. 20, 2022

(30) Foreign Application Priority Data
Jun. 30, 2020 (JP) 2020-112482

(51) Int. Cl. C12N 5/0783

(52) U.S. Cl. CPC C12N 5/0637; C12N 2529/10

(57) ABSTRACT

To provide a method of inducing regulatory T cells without the use of additives, disclosed herein is a method of inducing regulatory T cells comprising: a step of extracting blood from a living body; and a step of irradiating the extracted blood with ultraviolet light having a wavelength ranging between 260 nm and 320 nm. By irradiating the blood with ultraviolet light in the above specific wavelength range, it makes it possible for regulatory T cells to be induced without adding any substance to the blood extracted from a living body.

### TECHNICAL FIELD

The present invention relates to a method of inducing regulatory T cells in blood.

### BACKGROUND ART

The human body is equipped with immunity as a mechanism to prevent invasion from foreign substances and viruses from outside the body. The immunity involves effector T cells, which are responsible for defense against foreign invaders, and regulatory T cells, which regulate the function of the effector T cells in a suppressive direction, and a balance between the effector T cells and the regulatory T cells is critical. It has been known that autoimmune diseases and allergic diseases such as hay fever are caused by an imbalance in the above balance, whereby the effector T cells become more active relative to the regulatory T cells.

Therefore, in recent years, research has been conducted to induce regulatory T cells for the treatment of autoimmune and allergic diseases (see, for example, Patent Literatures 1 to 3).

### LISTING OF REFERENCES

### Patent Literature

PATENT LITERATURE 1: International Publication of PCT International Application WO2018/117090 A
PATENT LITERATURE 2: Laid-open Publication of Japanese Patent Application No. 2019-080497 A
PATENT LITERATURE 3: Laid-open Publication of Japanese Patent Application No. 2019-058182 A

### SUMMARY OF THE INVENTION

### Problems to Be Solved by the Invention

However, all of the methods disclosed in the above-mentioned patent literatures induce the regulatory T cells by administering additives (e.g., stimulants) to the blood. It may raise concerns about adverse effects of such additives on the human body in respects other than induction of the regulatory T cells.

The present invention has been made in order to solve the above mentioned problems and an object thereof is to provide a method of inducing regulatory T cells in the blood without the use of additives.

### Solution to Problems

In order to solve the above mentioned problems, according to one aspect of the present invention, there is provided a method of inducing regulatory T cells comprising: a step of extracting blood from a living body; and a step of irradiating the extracted blood with ultraviolet light having a wavelength ranging between 260 nm and 320 nm.

Thus, it makes it possible to induce the regulatory T cells in blood solely by irradiating blood extracted from a living body (e.g., a human body) with ultraviolet light in the specific wavelength range described above. In other words, it makes it possible to induce the regulatory T cells in blood without using any additives. It should be noted that inducing regulatory T cells means increasing the ratio of the regulatory T cells (CD25-positive, Foxp3-positive cells) in helper T cells (CD4-positive cells) in the blood.

In the above method of inducing regulatory T cells, the step of irradiating may irradiate the extracted blood with ultraviolet light having a wavelength ranging between 260 nm and 290 nm.

In this case, it makes it possible to induce the regulatory T cells even with a relatively small amount of ultraviolet irradiation dose. As a result, it makes it possible to shorten the irradiation time of the ultraviolet light.

Furthermore, the above method of inducing regulatory T cells may further comprise: a step of cultivating the blood that has been irradiated with the ultraviolet light for at least two days.

In this case, it makes it possible to appropriately induce the regulatory T cells in the blood.

Yet furthermore, the above method of inducing regulatory T cells may further comprise: a step of introducing the extracted blood into a flow channel, wherein the step of irradiating may include a step of irradiating the blood introduced into the flow channel with the ultraviolet light through a wall of the flow channel, and a length of an irradiation direction of the ultraviolet light may range between 70 µm and 500 µm.

Thus, by introducing the blood into a flow channel of which length in an irradiation direction of the ultraviolet light is slightly larger than that of the leukocyte, it makes it possible to create an environment in which leukocytes are easily aligned along the longitudinal direction (i.e., transfusion direction) of the flow channel. Therefore, it makes it possible to appropriately irradiate the leukocytes with the ultraviolet light that has transmitted through the wall of the flow channel. As a result, it makes it possible to eliminate complicated blood cell separation processes, such as separating only the leukocytes from the blood and irradiating the separated leukocytes with the ultraviolet light.

### Advantageious Effect of the Invention

According to the present invention, it makes it possible to induce regulatory T cells without the use of additives.

The above mentioned and other not explicitly mentioned objects, aspects and advantages of the present invention will become apparent to those skilled in the art from the following embodiments (detailed description) of the invention by referring to the accompanying drawings and the appended claims.

### BRIEF DESCRIPTION OF DRAWINGS

FIG. 1 is a schematic diagram illustrating an exemplary configuration of an ultraviolet irradiation system according to the present embodiment of the present invention.

FIG. 2 is a schematic diagram schematically illustrating an exemplary configuration of an ultraviolet irradiation apparatus according to the present embodiment of the present invention.

FIG. 3 is a diagram explaining a graph representing the degree of positivity of Foxp3 and CD25.

FIG. 4A is a diagram illustrating the changes in regulatory T cells after being irradiated with ultraviolet light.

FIG. 4B is a diagram graphically depicting the ratio of regulatory T cells.

FIG. 5 is a graph illustrating the wavelength dependency of ultraviolet light related to an increase in regulatory T cells.

### DESCRIPTION OF EMBODIMENTS

Hereinafter, non-limiting embodiments of the present invention will be described in detail with reference to the accompanying drawings.

The present embodiment will describe a method of inducing regulatory T cells in blood.

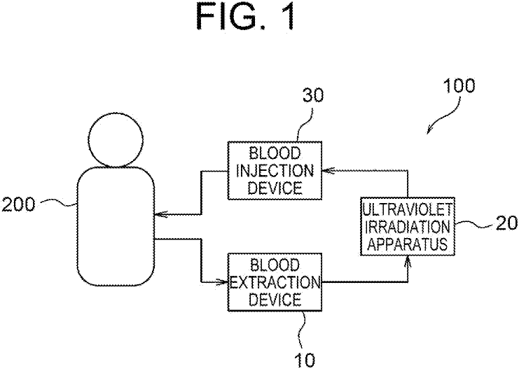

FIG. 1 is a schematic diagram illustrating an exemplary configuration of an ultraviolet irradiation system 100 according to the present embodiment.

The ultraviolet irradiation system 100 is a system that extracts blood from a living body (e.g., a human body such as a patient) 200, irradiates the extracted blood with ultraviolet light in a specific wavelength range to induce regulatory T cells, and thereafter returns the irradiated blood to the human body 200.

Here, inducing regulatory T cells means increasing the ratio of the regulatory T cells in the blood, more specifically the ratio of the regulatory T cells among CD4-positive cells, which in turn includes increasing the number of the regulatory T cells and decreasing the number of effector T cells.

As shown in FIG. 1, the ultraviolet irradiation system 100 includes a blood extraction device 10, an ultraviolet irradiation apparatus 20, and a blood injection device 30.

The blood extraction device 10 extracts blood from the human body 200 and supplies the extracted blood to the ultraviolet irradiation apparatus 20. The blood supplied to the ultraviolet irradiation apparatus 20 is blood from which the blood components have not been separated (i.e., whole blood).

The ultraviolet irradiation apparatus 20 irradiates the blood extracted by the blood extraction device 10 with ultraviolet light having the wavelength ranging between 260 nm and 320 nm. Details of the specific configuration of the ultraviolet irradiation apparatus 20 will be described below.

The blood injection device 30 returns the blood that has been irradiated with the ultraviolet light by the ultraviolet irradiation apparatus 20 to the human body 200.

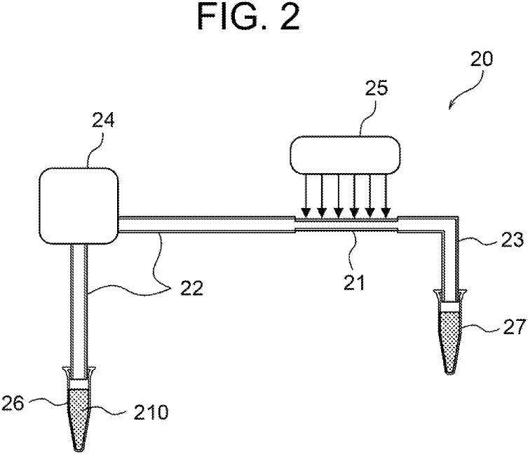

FIG. 2 is a schematic diagram schematically illustrating an exemplary configuration of the ultraviolet irradiation apparatus 20.

The ultraviolet irradiation apparatus 20 includes a flow channel 21, an inlet side transfusion tube 22, an outlet side transfusion tube 23, a transport pump 24, a light source 25, a supply container 26, and a collection container 27.

The flow channel 21 is a narrow tube made of a UV-permeable material into which the blood extracted by the blood extraction device 10 is introduced. The flow channel 21 is, for example, a quartz narrow tube. The flow channel 21 may be a narrow tube having a cylindrical shape with, for example, the length of 50 mm, an outer diameter of 3 mm, and an inner diameter of 0.3 mm (i.e., 300 µm).

The inlet side transfusion tube 22 is attached to the inlet side of the flow channel 21, and the outlet side transfusion tube 23 is attached to the outlet side of the flow channel 21. Each of the transfusion tubes 22 and 23 may be made of silicone tubing.

The transport pump 24 is interposed in the inlet side transfusion tube 22. The transport pump 24 is made of, for example, a peristaltic pump. The transport pump 24 allows the blood 210 to flow from the inlet side transfusion tube 22 to the outlet side transfusion tube 23 via the flow channel 21 at a preset flow velocity.

The light source 25 is disposed above the flow channel 21. The light source 25 may be an LED irradiator that irradiates light including ultraviolet light. More specifically, LEDs of the LED irradiator 25 emit light including ultraviolet light having the wavelength ranging between 260 nm to 320 nm, preferably 260 nm to 290 nm. The ultraviolet light emitted from the light source 25 is irradiated onto the flow channel 21, passes through the wall of the flow channel 21, and then irradiated onto the blood in the flow channel 21.

The light source 25 is not limited to the LED irradiator, but may be a XeCl excimer discharge lamp, a metal halide lamp, a fluorescent lamp, a mercury lamp, and the like. Also, the light source 25 may be a linear light source or a planar light source.

The light source 25 may be equipped with an optical filter that transmits only ultraviolet light having the wavelength ranging between 260 nm and 320 nm, preferably between 260 nm and 290 nm, and blocks light in all other wavelength ranges among light emitted from the LED elements or the lamp.

The supply container 26 is configured to contain the blood 210 extracted by the blood extraction device 10. The inlet of the inlet side transfusion tube 22 is inserted into the supply container 26.

The outlet of the outlet side transfusion tubing 23 is inserted into the collection container 27. The collection container 27 collects the blood 210 that has been irradiated with the ultraviolet light in the flow channel 21 transported through the outlet side transfusion tube 23.

Hereinafter, the fundamental operation of the ultraviolet irradiation apparatus 20 will be described.

The ultraviolet irradiation apparatus 20 operates the transport pump 24 in a state in which the inlet of the inlet side transfusion tube 22 is inserted into the supply container 26 containing the blood 210 extracted by the blood extraction device 10 and the outlet of the outlet side transfusion tube 23 is inserted into the collection container 27. Subsequently, the inlet side transfusion tube 22 sucks up the blood 210 into the supply container 26.

The blood 210 sucked up into the inlet side transfusion tube 22 is then introduced into the flow channel 21 made of a quartz narrow tube. The blood 210 flows through the flow channel 21 at a preset velocity.

Subsequently, the ultraviolet irradiation apparatus 20 turns on LEDs of the light source 25, which is constituted with the LED irradiator, allows the emitted ultraviolet light to transmit through the wall of the flow channel 21, and irradiates the blood 210 introduced into the flow channel 21 with the transmitted ultraviolet light.

The blood 210, which has been irradiated with the ultraviolet light in the flow channel 21, is transported to the outlet side transfusion tube 23 and then collected in the collection container 27 through the outlet side transfusion tube 23.

The blood 210 collected in the collection container 27 is then transported to the blood injection device 30 and returned to the human body 200 by the blood injection device 30.

It should be noted that, although the present embodiment describes a certain case in which the flow channel 21 is a narrow tube made of quartz glass, alternatively, the flow channel 21 may be a narrow tube made of glass, such as alkali glass, borosilicate glass, or the like. Furthermore, the flow channel 21 may also be a narrow tube made of synthetic resins such as silicone resin, cyclo-olefin resin (such as cyclo-olefin polymer (COP) and cyclo-olefin copolymer (COC)), acrylic resin, or the like.

The shape of the flow channel 21 is not limited to the shape described above.

The length of the flow channel 21 in the longitudinal direction (i.e., in the transfusion direction of the blood 210) may be set appropriately such that the irradiation dose of the ultraviolet light (i.e., the amount of the ultraviolet irradiation) onto the blood 210 in the flow channel 21 becomes to be the desired amount of ultraviolet irradiation, based on the intensity of the ultraviolet light emitted from the light source 25 or the flow velocity of the blood 210 flowing in the flow channel 21.

The thickness of the wall of the flow channel 21 may be set appropriately according to the ultraviolet transmittance of the material that constitutes the flow channel 21.

The length of the flow channel 21 in the ultraviolet irradiation direction (i.e., vertical direction in FIG. 2) may be within the range between 70 µm and 500 µm, which slightly larger than the leukocyte. The length of the flow channel 21 in the ultraviolet irradiation direction may be preferably between 100 µm and 300 µm, and more preferably between 200 µm and 300 µm.

Furthermore, the shape of the flow channel 21 is not limited to a cylindrical shape with only one transfusion channel through which the blood 210 passes, but alternatively, may be a rod body with multiple transfusion channels that are independent from one another. In this case, the multiple transfusion channels in the rod body and the light source 25 are arranged such that all transfusion channels are directly irradiated with the ultraviolet light without being intervened by other transfusion channels.

The shape of the transfusion channel through which the blood 210 passes may be any shape. When the shape of the transport channel is rectangular in cross section, the length in the ultraviolet irradiation direction is to be between 70 µm and 500 µm, which is slightly larger than that of the leukocyte as described above. In addition, in order to create an environment in which leukocytes can be readily aligned in the longitudinal direction, the length in the so-called width direction, which is orthogonal to the ultraviolet irradiation direction, is also to be between 70 µm and 500 µm, which is slightly larger than that of the leukocyte.

As described above, by irradiating the blood 210 extracted from the human body 200 with light including ultraviolet light having the wavelength ranging between 260 nm to 320 nm, it makes it possible to induce regulatory T cells in the blood 210 without using any additives such as stimulants.

Recent studies have revealed the facts that the regulatory T cells are positive for both the factor of Foxp3 and the factor of CD25 and that a cell in question is a regulatory T cell as long as the cell is positive for both Foxp3 and CD25.

In addition, using a flow cytometer (e.g., Fluorescence Activated Cell Sorting (FACS)), it is possible to depict how many cells that are positive for both Foxp3 and CD25, i.e., regulatory T cells, are present with respect to the total cells in the sample to be investigated.

The flow cytometers are devices that are able to examine the function and state of cells based on scattering and fluorescence.

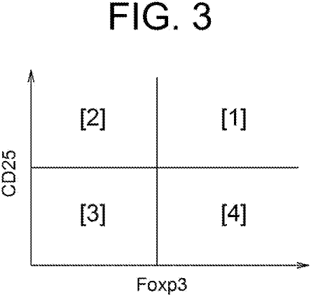

For example, the flow cytometer, as shown in FIG. 3, is capable of generating a graph with the degree of positivity for Foxp3 on the horizontal axis and the degree of positivity for CD25 positivity on the vertical axis. This graph may be divided into four regions [1] to [4] as shown in FIG. 3. Here, the upper right region [1] is positive for both Foxp3 and CD25, and thus the cells in this region [1] may be determined to be the regulatory T cells.

It should be noted that the upper left region [2] in FIG. 3 is negative for Foxp3 and positive for positive for CD25, and the lower left region [3] is negative for both Foxp3 and CD25. The lower right region [4] in FIG. 3 is positive for Foxp3 and negative for CD25.

### Experiment 1

The following experiments were conducted to confirm the changes in regulatory T cells in blood after being irradiated with the ultraviolet light using the method according to the present invention.

A circulation system similar to the ultraviolet irradiation apparatus 20 shown in FIG. 2 was constructed, and the sample blood 210 was flowed into the circulation system. Human peripheral blood diluted to 20% in phosphate buffered saline was used as the sample blood 210.

When the sample blood 210 was passing through the flow channel 21, which is a quartz narrow tube section, the sample blood 210 was irradiated with the ultraviolet light emitted from the light source 25, which is an LED irradiator. The irradiation wavelength was set to 290 nm. The flow velocity of the sample blood 210 was set such that the irradiation dose of the ultraviolet light in the flow channel 21 was 10 mJ/cm².

The group of the sample blood 210 that solely passed through the above circulation system without being irradiated with the ultraviolet light was used as the control group to be compared.

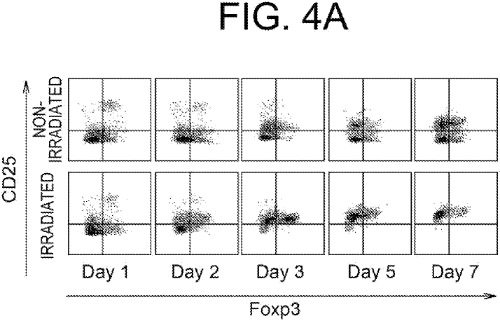

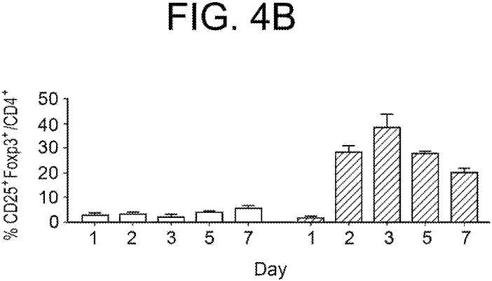

The UV-irradiated sample blood 210 and non-UV-irradiated sample blood 210 were collected, respectively, added to the culture medium, and then cultivated for 1, 2, 3, 5, and 7 days at 37° C. and under 5% CO₂, respectively. Subsequently, CD4-positive cells were gated from whole blood by the FACS analysis and changes in CD25 and Foxp3 were examined. The results thereof are shown in FIGS. 4A and 4B.

The upper row of FIG. 4A denotes the results for the non-UV-irradiated samples, and the lower row of FIG. 4A denotes the results for the UV-irradiated samples. The results are shown after 1, 2, 3, 5, and 7 days after being irradiated with the ultraviolet light, respectively. In this graph of FIG. 4A, each of dots corresponds to one cell.

FIG. 4B is a diagram graphically illustrating the ratio of the regulatory T cells (i.e., cells positive for both Foxp3 and CD25: cells in the region [1] in FIG. 3). The white-out bars denote the results for samples that were not irradiated with the ultraviolet light, and the shaded bars denote the results for samples that were irradiated with the ultraviolet light. Values in FIG. 4B are mean values for N = 3, and error bars indicate standard deviations.

As apparent from the results in FIG. 4B, in the case of non-UV-irradiated samples, although the ratio of the regulatory T cells increased slightly due to the cultivation, the ratio of the regulatory T cells never exceeded 10%. On the other hand, in the case of UV-irradiated samples, the ratio of the regulatory T cells increased from the second day of cultivation and reached its peak (approximately 40%) in three days.

Thus, for the UV-irradiated samples, as compared to the non-UV-irradiated samples, it was confirmed that the number of the regulatory T cells (i.e., cells positive for both Foxp3 and CD25) significantly increases after two days of cultivation and beyond. In other words, it was confirmed that irradiating blood with the ultraviolet light can be expected to increase the number of the regulatory T cells.

### Experiment 2

The following experiments were conducted to confirm the wavelength dependency of ultraviolet light to be irradiated onto blood related to the increase in the regulatory T cells.

A circulation system similar to the ultraviolet irradiation apparatus 20 shown in FIG. 2 was constructed, and the sample blood 210 was flowed into the circulation system. Human peripheral blood diluted to 20% in phosphate buffered saline was used as the sample blood 210.

When the sample blood 210 was passing through the flow channel 21, which is a quartz narrow tube section, the sample blood 210 was irradiated with the ultraviolet light emitted from the light source 25, which is an LED irradiator. The irradiation wavelength was set to 260 nm, 290 nm, 310 nm, and 365 nm, respectively. The flow velocity of the sample blood 210 was set such that the irradiation dose [mJ/cm²] of the ultraviolet light in the flow channel 21 was to be one of the irradiation doses shown in Table 1 below.

TABLE. 1

| WAVELENGTH [nm] | IRRADIATION DOSE [mJ/cm²] | | | |
|---|---|---|---|---|
| 260 | 0.1 | 1 | 10 | |
| 290 | 0.1 | 1 | 10 | 100 |
| 310 | 0.1 | 1 | 10 | 68 |
| 365 | 10 | 100 | 1200 | 10000 |

The UV-irradiated blood samples 210 were collected, added to the culture medium, and cultivated for two days at 37° C. and under 5% CO₂, respectively. According to the results of Experiment 1, as the significant induction effects of regulatory T cells was observed after two days of cultivation from being irradiated with the ultraviolet light, thus the samples from that day was used.

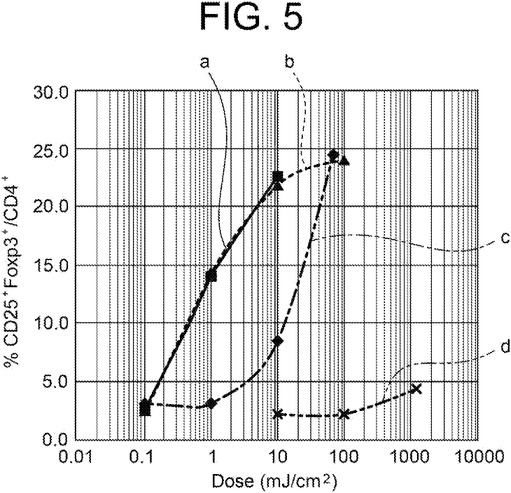

Subsequently, CD4-positive cells were gated from whole blood by the FACS analysis, and the ratio of CD25-positive and Foxp3-positive cells was examined. The results thereof are shown in FIG. 5.

In FIG. 5, the horizontal axis denotes the irradiation dose of ultraviolet light [mJ/cm²] and the vertical axis denotes the ratio of the regulatory T cells with respect to total cells in the sample [%].

Referring to FIG. 5, the curve a (filled square) denotes the result at the wavelength of 260 nm, the curve b (filled triangle) denotes the result at wavelength of 290 nm, the curve c (filled diamond) denotes the result at the wavelength of 310 nm, and the curve d (x-mark) denotes the result at the wavelength of 365 nm, respectively.

Normally, the ratio of regulatory T cells ranges between 3% and 8%. Therefore, when the ratio of the regulatory T cells is 10% or more, then it can be said to be effective in inducing the regulatory T cells.

In the case of the wavelength of 365 nm, when irradiating with the irradiation dose from 10 mJ/cm² to 1200 mJ/cm², the ratio of the regulatory T cells hardly increased and no induction effect was observed. In addition, when irradiating with the irradiation dose of 10000 mJ/cm², the irradiation could not be completed because the blood coagulated during the ultraviolet irradiation to form a thrombus.

On the other hand, in the case of the wavelength of 310 nm, although in the region of the irradiation dose of 10 mJ/cm² or less, the ratio of the regulatory T cells is less than 10%, in the region where the irradiation dose exceeds 10 mJ/cm², the ratio of the regulatory T cells exceeds 10%, which indicates the affirmative effects of induction.

Furthermore, in the cases of the wavelengths of 290 nm and 260 nm, both at the irradiation dose of 1 mJ/cm², the ratio of the regulatory T cell percentage exceeds 10%, which also indicates the affirmative effects of induction.

Those results confirm that by irradiating with the ultraviolet light in the wavelength region ranging between 260 nm and 310 nm, the induction effects of regulatory T cells are obtainable. In the above experiments, as a representative UVB light source, a light source having the peak wavelength of 310 nm with the spectral half-width of approximately 20 nm was used. Ultraviolet light is classified into any of UVA, UVB, and UVC, and similar effects on living bodies are generally assumed to emerge within each of three categories. For this reason, since the induction effect was confirmed at the peak wavelength of 310 nm, it is likely that the same effect is expected to be obtainable up to the peak wavelength of 320 nm. Furthermore, in the case of the wavelengths from 260 nm to 290 nm, it was confirmed that the induction effect of regulatory T cells was obtained even with a small irradiation dose. In other words, by using the ultraviolet light having the wavelength ranging between 260 nm and 290 nm, it makes it possible to shorten the irradiation time of the ultraviolet light.

As described above, the method of inducing regulatory T cells according to the present embodiment includes a step of extracting blood from a living body (e.g., human body) and a step of irradiating the extracted blood with ultraviolet light in a specific wavelength range. Here, the ultraviolet light to be irradiated onto the blood includes ultraviolet light having the wavelength ranging between 260 nm and 320 nm.

In this way, by using UVB or UVC ultraviolet light, which have relatively high light energy, it makes it possible to induce regulatory T cells in blood without using any additives such as stimulants, simply by irradiating the blood extracted from the human body with such ultraviolet light.

Nucleic acids (DNA) are known to have an absorption peak at the wavelength around 260 nm. Furthermore, erythrocytes (i.e., hemoglobin) are known to absorb more light at shorter wavelengths than 260 nm. Blood contains more erythrocytes than leukocytes. Thus, when the ultraviolet light irradiated onto blood is absorbed by erythrocytes, leukocytes, which are the original target, are unlikely to be irradiated with the ultraviolet light, thereby the effects of inducing regulatory T cells are hardly obtainable.

According to the present embodiment, with the lower limit of the wavelength being 260 nm, the blood is irradiated with the ultraviolet light having the wavelength equal to or greater than 260 nm. As a result, it makes it possible to irradiate blood with ultraviolet light with high absorption by DNA and low absorption by hemoglobin so as to obtain the effects of inducing regulatory T cells appropriately.

Furthermore, by setting the ultraviolet light to be irradiated onto the blood to be within the wavelength ranging between 260 nm and 290 nm, as shown in FIG. 5, it makes it possible to certainly obtain the effects of inducing regulatory T cells even with a relatively small irradiation dose, thereby shortening the irradiation time of the ultraviolet light.

It should be noted that, when the wavelength of the ultraviolet light irradiated onto blood exceeds 290 nm (e.g., 310 nm), as shown in FIG. 5, the irradiation dose of the ultraviolet light is required to be equal to or greater than 20 mJ/cm². Since the method according to the present embodiment does not irradiate blood with the ultraviolet light through the skin of the human body as in commonly used conventional ultraviolet light therapy devices, but extracts (i.e., separates) blood from the human body and then irradiates the extracted blood with the ultraviolet light outside the human body, it makes it possible to readily set the irradiation dose of the ultraviolet light to the desired irradiation dose as appropriate.

Yet furthermore, the method of inducing regulatory T cells according to the present embodiment may include a step of cultivating blood that has been irradiated with the ultraviolet light in the specific wavelength range described above for two or more days. By cultivating the blood irradiated with the ultraviolet light for two or more days, it makes it possible to appropriately induce the regulatory T cells in the blood.

It should be noted that the blood irradiated with the ultraviolet light may be returned directly to the living body without being cultivated. In this case, it is expected to appropriately induce the regulatory T cells in the blood in the living body as well.

Yet furthermore, according to the method of inducing regulatory T cells of the present embodiment, as described above, the blood extracted from the human body may be introduced into the flow channel 21 of which length in the irradiation direction of ultraviolet light is between 70 µm and 500 µm, and the blood introduced into the flow channel 21 may be irradiated with the ultraviolet light, which has transmitted through the wall of the flow channel 21.

In this way, by setting the size of the flow channel 21 into which blood is introduced to be slightly larger than the size of the leukocyte, it makes it possible to create an environment in which leukocytes can be readily aligned along the longitudinal direction of the flow channel 21 (i.e., blood transfusion direction). For this reason, it makes it possible to suppress the ultraviolet light transmitted through the wall of the flow channel 21 from being absorbed by erythrocytes in the blood flowing in the flow channel 21 and to irradiate the leukocytes with the ultraviolet light appropriately. As a result, it makes it possible to eliminate complicated blood cell separation processes, such as separating leukocytes from erythrocytes in the blood by the centrifugal separation, extracting only the leukocytes, and irradiating the extracted leukocytes with the ultraviolet light.

As described above, according to the present embodiment, it makes it possible to induce regulatory T cells in the blood solely by the ultraviolet irradiation, without requiring complicated processes such as centrifugation, and without the use of any additives such as stimulants.

Although specific embodiments have been described above, the embodiments described are illustrative only and are not intended to limit the scope of the present invention. The apparatus and method described herein may be embodied in other forms than as described above. In addition, it is also possible to appropriately omit, substitute, or modify the above described embodiments without departing from the scope of the present invention. Embodiments with such omissions, substitutions and modifications fall within the scope of the appended claims and equivalents thereof and also fall within the technical scope of the present invention.

### REFERENCE SIGNS LIST

10: Blood Extraction Device; 20: Ultraviolet Irradiation Apparatus; 21: Flow Channel; 22: Inlet Side Transfusion Tube; 23: Outlet Side Transfusion Tube; 24: Transport Pump; 25: Light Source; 26: Supply Container; 27: Collection Container; 30: Blood Injection Device; 100: Ultraviolet Irradiation System; 200: Human Body; 210: Blood

1. A method of inducing regulatory T cells comprising:
a step of extracting blood from a living body; and
a step of irradiating the extracted blood with ultraviolet light having a wavelength ranging between 260 nm and 320 nm.

2. The method of inducing regulatory T cells according to claim 1, wherein
the step of irradiating irradiates the extracted blood with ultraviolet light having a wavelength ranging between 260 nm and 290 nm.

3. The method of inducing regulatory T cells according to claim 1, further comprising:
a step of cultivating the blood that has been irradiated with the ultraviolet light for at least two days.

4. The method of inducing regulatory T cells according to claim 1, further comprising:
a step of introducing the extracted blood into a flow channel, wherein
the step of irradiating includes a step of irradiating the blood introduced into the flow channel with the ultraviolet light through a wall of the flow channel, and
a length of an irradiation direction of the ultraviolet light ranges between 70 µm and 500 µm.
